(19) United States
(12) Patent Application Publication
(10) Pub. No.: US 2020/0143914 A1
(43) Pub. Date: May 7, 2020

(54) User Interface, System, and Method for Optimization of Patient Problem List Encoding

(71) Applicant: Intelligent Medical Objects, Inc., Northbrook, IL (US)

(72) Inventors: Jonathan Gold, Louisville, CO (US); John Tian, Gurnee, IL (US); David Arco, Chicago, IL (US); Steven Rube, Lake Forest, IL (US); Michael Decaro, Mount Prospect, IL (US); Joel Graff, Highland Park, IL (US)

(21) Appl. No.: 16/181,072

(22) Filed: Nov. 5, 2018

(51) Int. Cl. G16H 10/60; G06F 17/30

(52) U.S. Cl. CPC G16H 10/60; G06F 17/30289

(57) ABSTRACT

A system, method, and user interface for optimizing encoding of a patient problem list in an electronic medical record or electronic health record. The system is operable on one or more computers with one or more processors that are configured to construct a database of HCC signatures, each HCC signature including one or more ICD-10 and/or SNOMED codes, deconstruct a patient problem list within an electronic medical record or electronic health record to generate an identification of one or more ICD-10 and/or SNOMED codes included within the patient problem list, evaluate the identified ICD-10 and/or SNOMED codes against each HCC signature to determine sufficient matches to one or more HCC signatures, cross-check HCC concepts related to the sufficiently-mapped HCC signatures and eliminate HCC concepts already identified in the record to generate a group of at least one non-eliminated, sufficiently-mapped HCC concepts, and displaying the non-eliminated, sufficiently-mapped HCC concepts.

### BACKGROUND

### 1. Field of the Invention

The present application is directed to a user interface, a system, and a method for optimizing the encoding of patient problem lists within an electronic medical record or electronic health record.

### 2. Description of the Related Art

During a healthcare encounter, a patient may present with a multitude of different diseases, conditions, or other problems. Preferably, each of those problems gets recorded in the patient's electronic medical record (EMR) or electronic health record (EHR), e.g., in the patient's problem list. Ideally, that information is recorded in a way that captures the semantic meaning or clinical intent of the practitioner that entered and/or that is likely to review the information. Additionally or alternatively, that information may be entered in a standardized or normalized fashion, whereby entries from multiple practitioners for the same problem are entered identically. In either case, accurate entry of the patient's problems is important to provide a comprehensive record and to facilitate proper patient care.

At the same time, it is necessary to also have accurate data entry in the form of one or more external ontologies, which may serve different ancillary functions in the patient care process. Various ontologies have been developed for various reasons, including administrative code sets that may be designed to support administrative functions of healthcare, such as reimbursement and other secondary data aggregation; clinical code sets that encode specific clinical entities involved in clinical work flow and allow for meaningful electronic exchange and aggregation of clinical data for better patient care; and reference terminology code sets that may be considered a “concept-based, controlled medical terminology” to maintain a common reference point in the healthcare industry. Reference terminologies also identify relationships between their concepts, e.g., relationships can be hierarchically defined, such as a parent/child relationship.

Common examples of administrative code sets are the International Classification of Disease (ICD) and the Current Procedural Terminology, which is referred to via the trademark CPT. Examples of clinical code sets are the Logical Observation Identifiers Names and Codes, referred to under the trademark LOINC, and a normalized terminology for medication information, such as the terminology of the National Library of Medicine referred to under the trademark RxNorm. One example of a reference terminology is The Systematized Nomenclature of Medicine—Clinical Terms, referred to under the trademark “SNOMED CT.”

Although accurate data entry is necessary to accomplish both the clinical and ancillary functions, “accuracy” may not necessarily be the same thing in both contexts. For example, a patient record that includes “otitis media, resolved” may permit the practitioner to render the same degree of care as a record that includes “follow-up otitis media, resolved.” However, those two entries may be reflected by different ICD codes, such that one may be considered less “accurate” than the other depending on the facts surrounding the patient and his or her problem history. Aside from the type of care that may be rendered as a result of such recordkeeping, such distinctions may be significant when it comes to other aspects of the healthcare process, e.g., with regard to billing and insurance reimbursement.

In particular, certain entities such as the Centers for Medicare & Medicaid Services (“CMS”) may establish guidelines for reimbursement that depend on the underlying conditions being addressed. In particular, CMS has established a Hierarchical Condition Category (“HCC”) model to determine risk or seriousness of conditions. The HCC model relies on grouping approximately 9,000 to 10,000 of the almost 70,000 ICD-10-CM codes into a set of approximately 80 HCC Categories. Those categories then may be grouped into near-sequential groups of similar diseases or conditions. CMS then reimburses for conditions that are deemed less severe at a lower rate than those that are more severe. Thus, under that framework, accurate recordkeeping is desirable to ensure that the proper actions are being reimbursed at the proper rates.

What are needed are a system and method that preferably address one or more of these challenges.

### BRIEF SUMMARY

In one aspect, a method includes the steps of: translating a plurality of hierarchical condition category (HCC) codes into a plurality of HCC signatures, each HCC signature including one or more ICD-10 codes and/or one or more SNOMED codes; extracting a plurality of ICD-10 codes and/or SNOMED codes out of an electronic medical record or electronic health record; analyzing, using a comparison engine executed by a processor on a computer, the extracted codes against each of the HCC signatures; identifying at least one HCC signature for which a minimum number of ICD-10 codes and/or SNOMED codes appear in the electronic medical record or electronic health record; comparing the identified at least one HCC signature against HCC codes already recognized in the electronic medical record or electronic health record; and if the electronic medical record or electronic health record does not include the identified at least one HCC signature, updating the electronic medical record or electronic health record to recognize an HCC code associated with at least one of the identified HCC signatures.

In another aspect, the system is operable on one or more computers with one or more processors that are configured to execute instructions to construct a database comprising a plurality of HCC signatures, each HCC signature including one or more ICD-10 codes and/or one or more SNOMED codes, deconstruct a patient problem list within an electronic medical record or electronic health record to generate an identification of one or more ICD-10 and/or SNOMED codes included within the patient problem list, evaluate the identified one or more ICD-10 and/or SNOMED codes against each HCC signature to determine sufficient matches to one or more HCC signatures, cross-check HCC concepts related to the sufficiently-mapped one or more HCC signatures and eliminating HCC concepts already identified in the electronic medical record or electronic health record to generate a group of at least one non-eliminated, sufficiently-mapped HCC concepts, and display the non-eliminated, sufficiently-mapped HCC concept.

### BRIEF DESCRIPTION OF THE SEVERAL VIEWS OF THE DRAWINGS

FIG. 1 is a pseudo-entity relationship diagram outlining a process for optimizing a patient problem list and highlighting a process for generating a reference table from a plurality of HCCs;

FIG. 2 is a depiction of a master table of HCCs stored in a database;

FIG. 3 is a depiction of one entry in the master table of FIG. 2 and its associated interface terminology name and identifier;

FIG. 4 is an abstraction of an HCC concept mapping to items of a first external ontology and a second external ontology;

FIG. 5 is a depiction of the abstraction of FIG. 4 applied to the specific HCC identified in FIG. 3;

FIG. 6 is an abstraction of the generation of a parent table of first and second external ontology concepts resulting from the mappings of FIG. 4;

FIG. 7 is a depiction of the abstraction of FIG. 6 identifying each of the parent table components of the specific first and second external ontology concepts identified in FIG. 5;

FIG. 8 is a depiction of the generation of a specific parent table utilizing the components identified in FIG. 7;

FIG. 9 is the pseudo-entity relationship diagram of FIG. 1 highlighting a process for extracting first and second external ontology concepts from a patient problem list;

FIG. 10 is a depiction of a patient problem list table stored in a database;

FIG. 11 is a depiction of a process for identifying a plurality of first and second external ontology concepts from the table of FIG. 10 to generate an external ontology concept table;

FIG. 12 is a depiction of a process for identifying specific external ontology concepts relevant to one or more HCC concepts;

FIG. 13 is the pseudo-entity relationship diagram of FIG. 1 highlighting a portion of a workflow involving a comparison engine for analyzing the specifically-identified external ontology concepts of FIG. 12 against the reference table of FIG. 8;

FIG. 14 is a diagram highlighting a functional location and operation of the comparison engine identified in FIG. 13;

FIG. 15 is a diagram depicting operation of the comparison engine and potential outputs from the comparison engine; and

FIG. 16 is the pseudo-entity relationship diagram of FIG. 1 highlighting the operation of a display algorithm to generate a suggestion of HCC concepts to add to a patient problem list.

### DETAILED DESCRIPTION

As discussed in greater detail below and with reference to the accompanying figures, the present user interface, system, and method provide for analyzing a problem list of an electronic medical record or an electronic health record to identify and extract details sufficient to establish the existence of one or more Hierarchical Condition Categories (“HCC”s) and to determine whether that record should be updated to include an indication of those HCCs, thereby contributing to accuracy of the patient's record.

In another aspect, the present disclosure is related to a user interface, system, and method for determining if a patient's medical problems form an HCC or alter the level of a current HCC diagnosis. Clinically sound alternatives presented as the clinician reviews the patient's problem list can prompt the user for both greater diagnostic clarity and improved HCC documentation. Similarly, analytics that help an organization identify specific patients who may have unrecognized HCC diagnoses can streamline the review process.

In addition to complementing accurate patient recordkeeping, which may lead to better or more accurate treatment, the accurate capture of HCC conditions in an organization's patient population may directly affect a level of financial risk and reimbursement it faces. As patient diagnoses increase in complexity, greater investigation, review, and treatment are needed to ensure that accurate capture of relevant data. At the same time, doing so entails significant resource commitment and costs.

Construction of Parent Groups—“Signatures”

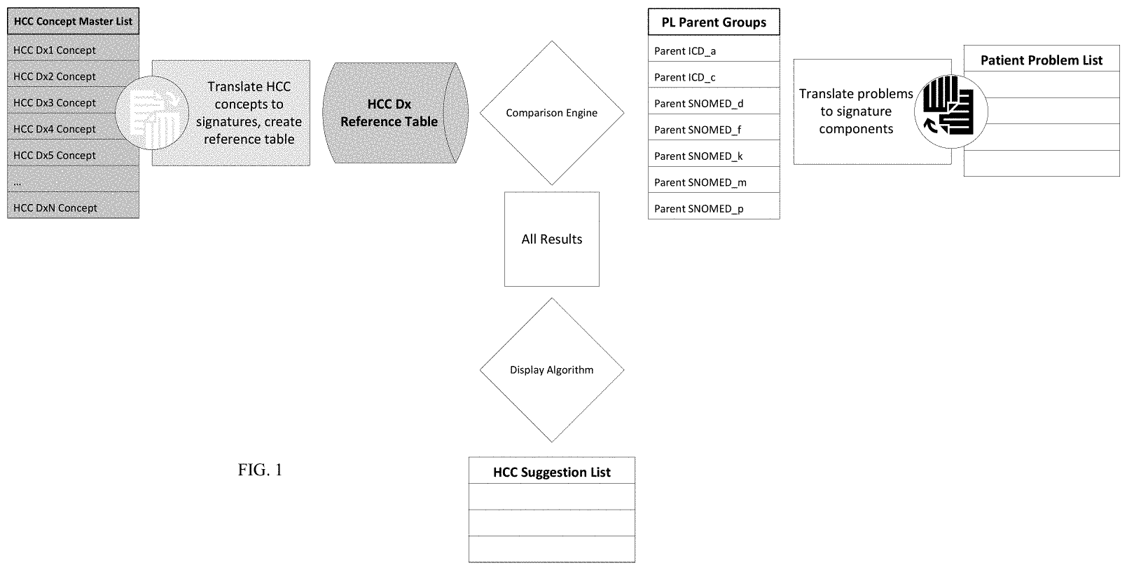

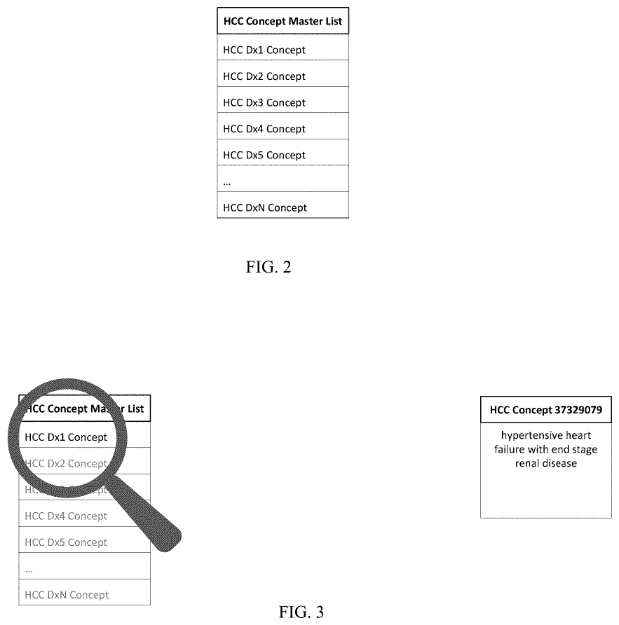

Turning to FIG. 1, the system first may analyze the HCC Concepts to generate one or more parent reference tables, i.e., one or more signature tables. For example, in one aspect, each HCC diagnosis or problem, i.e., an “HCC Concept,” may be represented by its own concept within an interface terminology, the concept including both a unique name and a unique alphanumeric identifier. A table of those groups, reflected as HCC Concepts, is depicted in FIG. 2. Currently there are 80+ such groups. As seen in FIG. 3, the exemplary HCC Concept Dx1 may be represented by the identifier 37329079 and the name “hypertensive heart failure with end stage renal disease.”

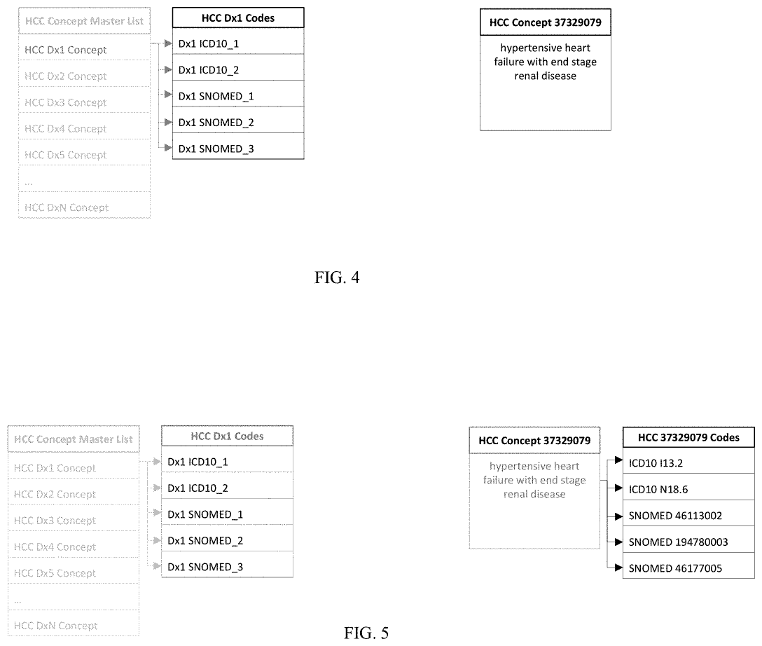

Turning now to FIG. 4, the HCC Concept further may be mapped to one or more codes of a first external ontology, e.g., ICD-10, and a second external ontology, e.g., SNOMED. For example, the HCC Concept 37329079 discussed above may map to ICD-10 codes 113.2 and N18.6, while also mapping to the SNOMED codes 46113002, 194780003, and 46177005, as seen in FIG. 5.

In the event that an ICD-10 structure is relied on to build a parent group, that group may be built by obtaining a current set of CMS-HCC diagnosis-related payment groups.

Similarly, in the event that a SNOMED hierarchy is used to build a parent group, the system may evaluate the specific SNOMED codes mapped to HCC concepts, as well as the parents of those concepts, to determine parallel “near miss” diagnostic groups.

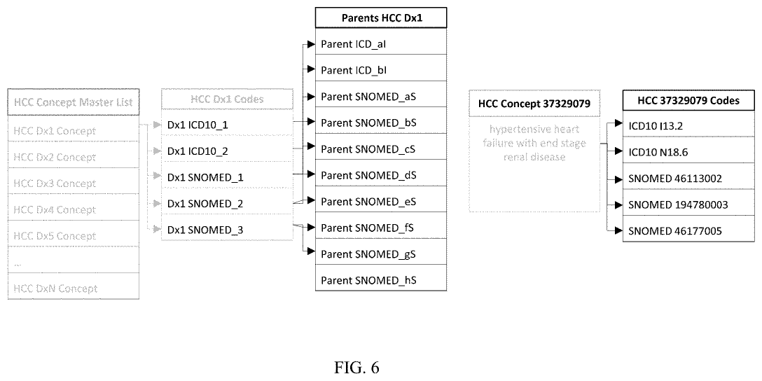

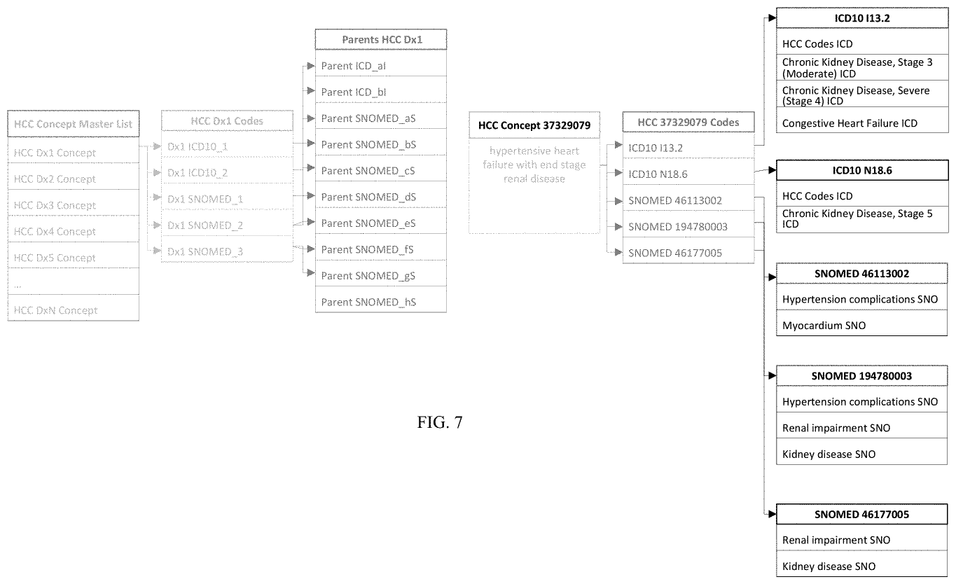

Turning to FIG. 6, one or more of the external ontologies may be organized in a hierarchical fashion, such that it is possible to construct a plurality of parent groups for each concept in the first and second ontologies, each parent group including a primary concept and one of more of the hierarchically-related child concepts. For example, as seen in FIG. 7, the ICD-10 and SNOMED codes that map to the HCC Concept each may be parents of or related to various other ICD and SNOMED codes. In this instance, the system may triangulate using multiple groups that may overlap on some codes in order to generate the list of first and second ontology concepts. For example, the code “ICD10 N18.6” may appear in a plurality of groups, including: (1) the specific HCC Concept being identified, (2) an HCC Concept that relates to a severity of the specific disease (in this instance, “end stage renal disease” may overlap with stage 5 chronic kidney disease (CKDS)), and (3) all similar diseases (e.g., all CKDs >2). Each of those groups may have other ICD or SNOMED codes besides ICD10 N18.6 associated with them, and the system may be configured to include all of those other related codes under the ICD10 N18.6 umbrella, even though they may not be formal children of that code.

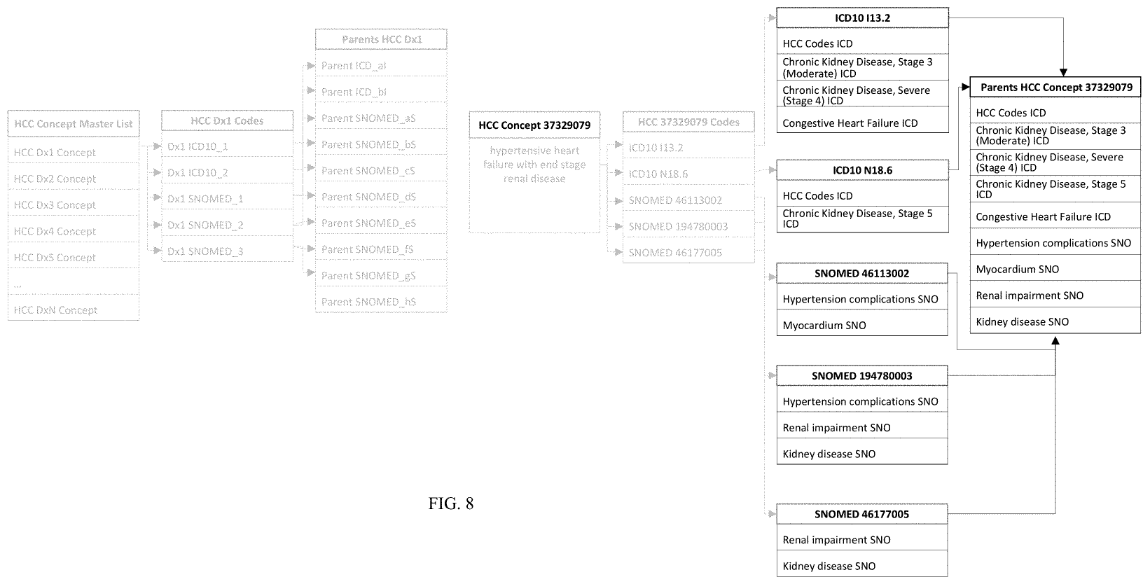

There may be overlap between several of the ICD-10 or SNOMED codes that map to the ICD-10 or SNOMED codes mapped to the HCC Concept. For example, both ICD-10 I13.2 and ICD-10 N18.6 may include maps to HCC Codes ICD. For each HCC Concept, the system may aggregate the non-duplicative codes to generate an HCC Concept Table, such as the Parents HCC Concept 37329079 table depicted in FIG. 8.

Once the HCC Concept—ICD-10/SNOMED mappings are obtained, those groups then may be expanded to include “near miss” diagnoses within groups. For example, CMS-HCC ICD-10 codes include Secondary hyperaldosteronism (E26.1), Bartter's syndrome (E26.81), Other hyperaldosteronism (E26.89), and Hyperaldosteronism, unspecified (E26.9). These ICD-10 codes appear in parent groups “Parent: HCC Codes_023 ICD” and “Parent: Endocrine disorders_Aldosterone, hyper ICD.” Additional ICD-10 codes, such as Primary hyperaldosteronism (E26.0), Conn's syndrome (E26.01), Glucocorticoid-remediable aldosteronism (E26.02), Other primary hyperaldosteronism (E26.09), and Other hyperaldosteronism (E26.8) may not be included in either of these CMS-HCC groups, but the system may add them to one or more of the ICD-10 parent groups, e.g., the “Parent: Endocrine disorders_Aldosterone, hyper ICD” group. In particular, the system may determine that, since multiple primary and secondary hyperaldosteronism codes are found in the HCC codes, there may be occasions where a clinician has selected a less specific concept or a highly specific one, when a more granular or a more general option, respectively, would be both a correct diagnostic choice AND capture the HCC association. In one aspect, these “near misses” may be determined by factoring in the original curation of the groups, as well as the ICD-10 and SNOMED hierarchies, along with a potentially variable factor relating to a match tightness requirement.

Each specific ontological concept mapped to the HCC Concept may be either a parent or child concept. However, that distinction may be immaterial when it comes to assigning the parent groups to each HCC code, i.e., the same parent group is assigned regardless of whether the HCC Concept maps to a parent or child concept within the external ontologies.

Each HCC Concept may map to a unique combination of codes of the first and second external ontologies and, as such, a unique set of parent groups. At the same time, however, there may be overlap with each in elements of the ontologies in that, e.g., an ICD-10 code or a SNOMED code can map to both a first HCC Concept and a second HCC Concept. Still further, a first HCC Concept may map to one or more ICD-10 or SNOMED codes, while a second HCC Concept may map to a parent or child of those codes. In either case, the first and second HCC Concepts then may have one or more parent groups in common. The following table illustrates these overlaps, where the problems identified in the top row header may be associated with one or more of an ICD-10 code and a SNOMED code:

| | Hypertensive kidney | Hypertensive | Hypertensive |
|---|---|---|---|
| | disease with end-stage | kidney disease, | kidney disease, |
| Parent Groups | renal disease | stage IV | stage V |
| Parent: Chronic Kidney | | x | |
| Disease_CKD1-4 ICD | | | |
| Parent: Chronic Kidney | | x | |
| Disease_CKD4 ICD | | | |
| Parent: Chronic Kidney | | x | x |
| Disease_CKD4-5 ICD | | | |
| Parent: Chronic Kidney | X | x | x |
| Disease_CKD4-ESRD ICD | | | |
| Parent: Chronic Kidney | | | x |
| Disease_CKD5 ICD | | | |
| Parent: Chronic Kidney | X | | |
| Disease_ESRD ICD | | | |
| Parent: End-Stage Renal Disease ICD | X | | |
| Parent: End-Stage Renal Disease SNO | x | | |
| Parent: Hypertension | x | x | x |
| complications SNO | | | |
| Parent: Hypertension | x | x | x |
| complications, kidney SNO | | | |
| Parent: Kidney disease SNO | x | x | x |
| Parent: Kidney | x | x | x |
| disease_Hypertensive SNO | | | |
| Parent: Kidney | x | x | x |
| disease_Renal impairment SNO | | | |
| Parent: Renal impairment SNO | x | x | x |
| Parent: Renal | x | | |
| impairment_Renal Failure SNO | | | |
| Parent: HCC Codes_136 ICD | x | | x |
| Parent: HCC Codes_137 ICD | | x | |

The inclusion of an external ontology code in multiple parent groups may serve both to boost fuzzy associations while aiding in triangulating on a more specific diagnosis if greater detail appears on the problem list. In other words, the similarities between the overlapping codes may serve to highlight the similarities between the HCC parent groups, while the non-overlapping, distinct codes may be used to determine which HCC parent group is most appropriate.

This process then may be repeated for each of the other HCC Concepts to thereby generate a plurality of HCC Concept parent tables.

The example provided above illustrates how different stages of a disease may be used to generate or determine different HCC parent groups. In another example, parent groups may be determined based on groupings surrounding systems or anatomy or body areas. For example, malignant neoplastic disease may have HCC parent groups surrounding severity, e.g., Parent: Malignant neoplastic disease, primary SNO and Parent: Malignant neoplastic disease, secondary SNO, while also having HCC parent groups relating to specific system or anatomic groups, e.g., Parent: Malignant neoplastic disease, connective tissue SNO, Parent: Malignant neoplastic disease, gastrointestinal SNO, and Parent: Malignant neoplastic disease, head/neck SNO; etc.

In still other instances, HCC parent groups may be constructed from more general categories (e.g., ‘Parent: Chronic Ischemic Heart Disease ICD’) or from more specific members of that general category (e.g., ‘Parent: Chronic Ischemic Heart Disease_Old MI ICD’). Use of more explicit parent groups boosts the relative ‘goodness-of-fit’ score that more closely resemble the diagnoses found on the problem list and diminishes those less likely candidate HCC's.

Construction of HCC Reference Table

Once parent groups have been constructed, the system may generate a table of HCC parent profiles for all HCC concepts. The resulting table may list each HCC Concept, as well as its associated parent group signature. For example, the table may include columns of the following information: (1) HCC Concept code, (2) the number of parent groups associated with a specific concept, (3) a group code, (4) a group title, (5) the number of times an HCC Concept's ICD-10 or SNOMED codes appear in a particular parent group, and (6) the highest ICD10 HCC factor for that concept based upon “CMS-HCC diagnosis factor.”

As discussed above, one of the aims of the system may be to identify HCC Concepts that are not formally identified in the patient's record, particularly those HCC Concepts that may result in more comprehensive CMS reimbursement. Those HCC Concepts tend to be more complex in nature or rely on a combination of multiple problems. For example, the HCC Concept “Hypertensive heart failure with end-stage renal disease” may include ICD-10 and/or SNOMED concepts relating to hypertension, heart disease, and renal disease, as well as additional concepts reflecting a severity of one or more of those problems. Thus, in one instance, the system may include an arbitrary or user-generated minimum cut-off in order to increase a rate of processing by reducing a size of the reference table and, accordingly, a number of possibilities against which the patient's record needs to be checked. For example, the system may exclude HCC Concepts that have fewer than ten parent groups from inclusion in the HCC reference table.

Translating Problem List

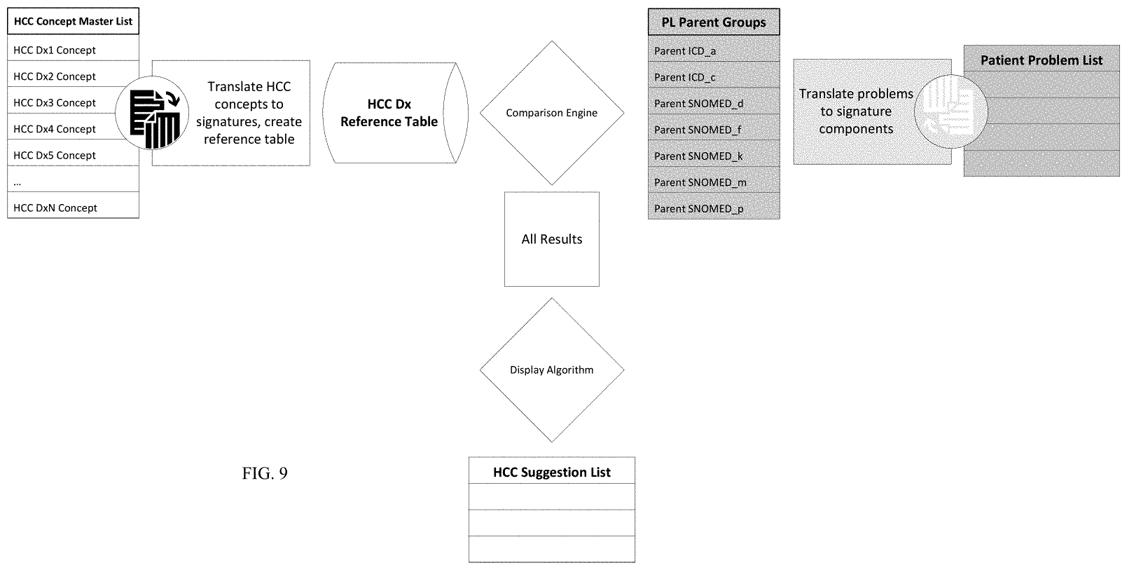

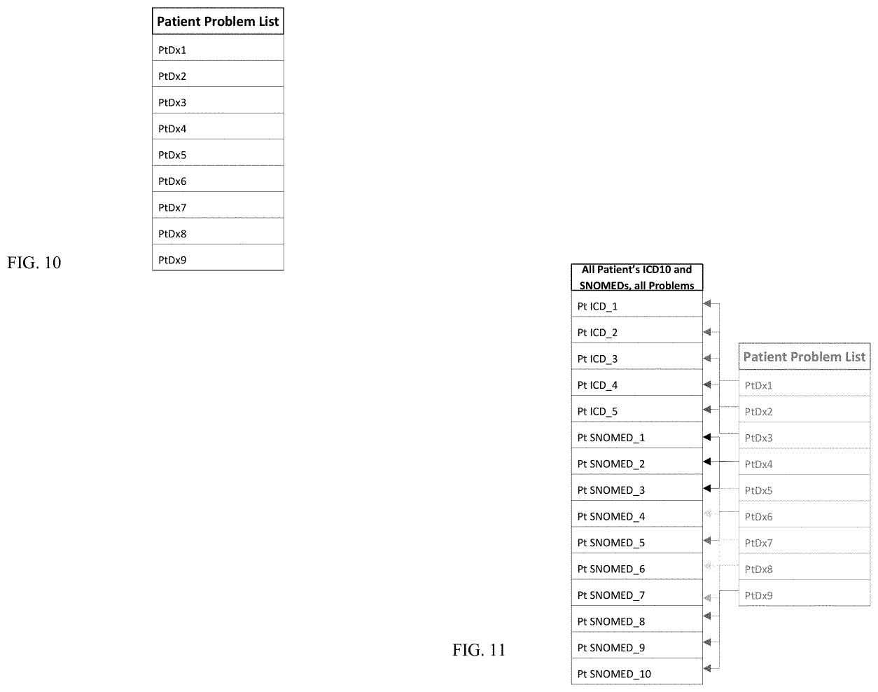

Turning now to FIG. 9, in order to evaluate a patient problem list to determine whether it may include potential additional HCC Concepts, the system may deconstruct the problem list by converting it into a list of all ICD-10 and SNOMED codes for all of its concepts and then determining which parent groups capture the many problem list codes. Thus, the system first may identify each entry in the patient's problem list, as at FIG. 10. From there, concepts within the first and second external ontologies, e.g., ICD-10 and SNOMED concepts, that map to each of the problem list entries may be extracted, as seen in FIG. 11. Those mappings may be determined directly, e.g., from known mappings between the problems and the ontologies. Alternatively, the problem list entries may be mapped to one or more interface terminology concepts in order to preserve the semantic meanings behind those problems, and the interface terminology concepts then may be mapped, either directly or via lexicals that are part of the interface terminology and that represent alternative ways to express the concepts, to each of the external ontology concepts. Examples of methods and utilities for doing such analysis and mapping may be found in the commonly-assigned U.S. patent application publications 2012/0179696, 2014/0122117, 2015/0242571, and 2016/0350362, the contents of each which are incorporated herein by reference in their entirety.

In various instances, the ICD-10 and SNOMED codes may not be associated with an HCC parent group. In those instances, the system may simply ignore those un-associated codes, which may reduce analysis time by optimizing the number of codes requiring comparison.

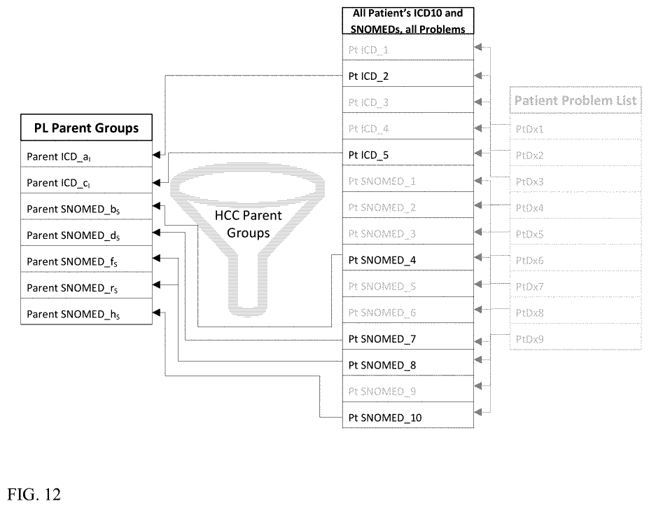

Once the ICD-10 and SNOMED mappings to the patient's problems have been identified, the system then may generate a problem list profile of all relevant parent groups, as seen in FIG. 12. In one aspect, a patient list parent group may comprise all ICD-10 and SNOMED codes that are present in the patient's problem list and that are known to map to one or more HCC Concepts. In another aspect, the system may include a plurality of pre-curated parent groups, e.g., reflecting groupings of ICD-10 and/or SNOMED codes that reflect more commonly-diagnosed problems.

Comparing a Problem List with the HCC Reference Table

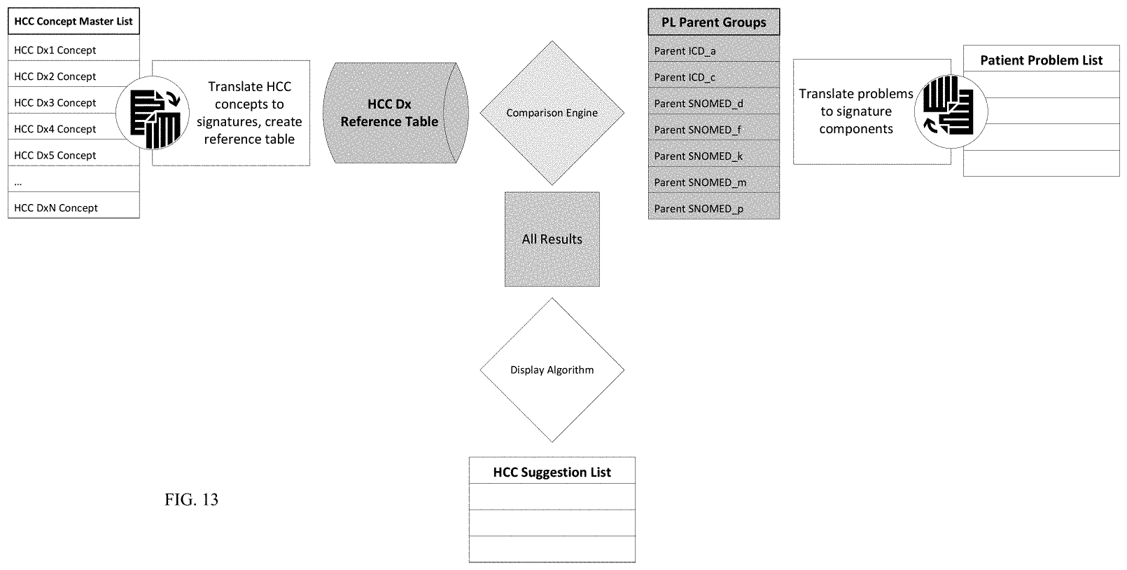

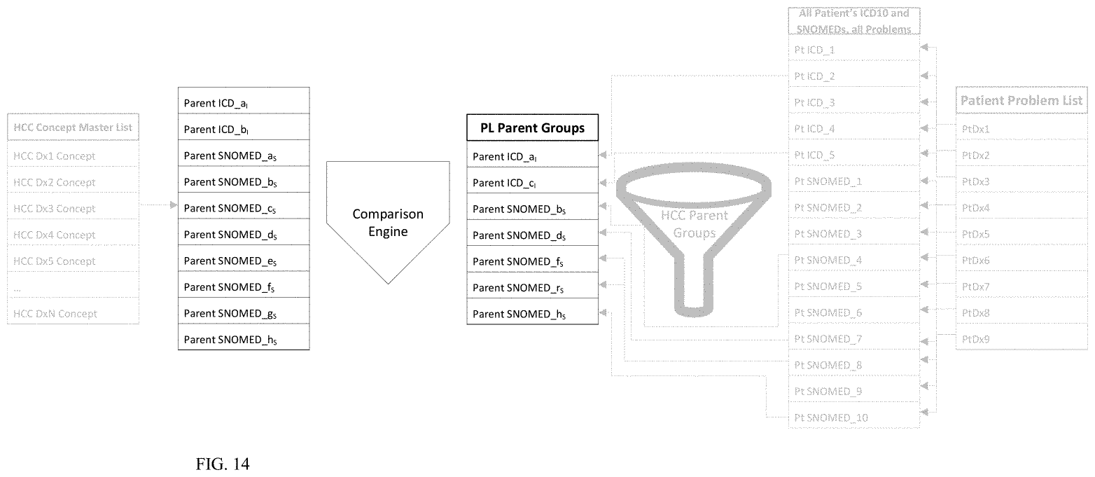

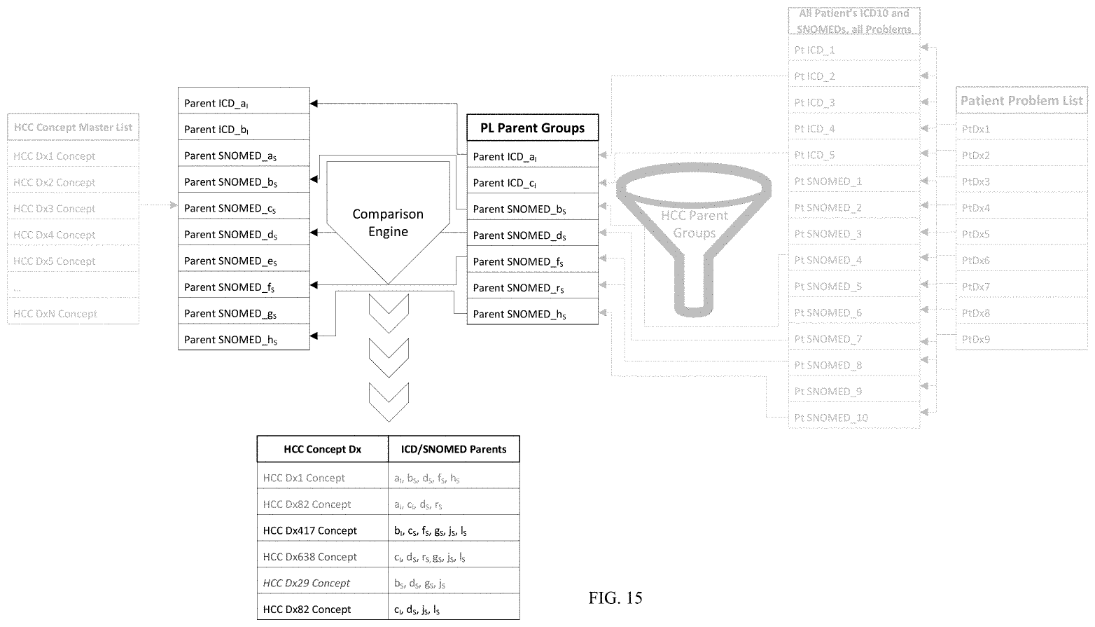

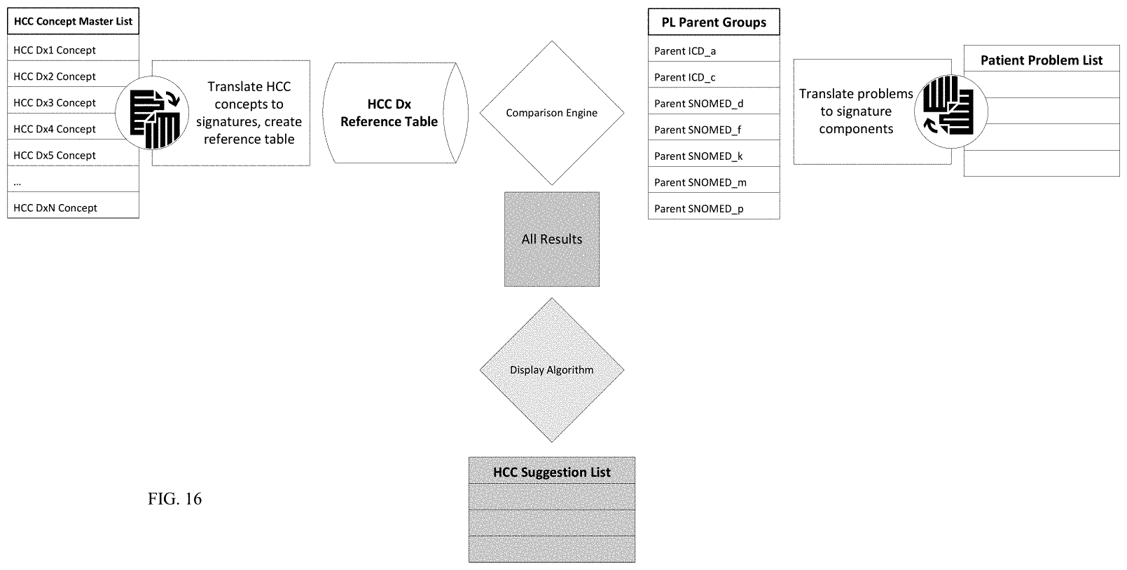

Turning now to FIGS. 13-16, once the codes have been associated to the parent groups, the system may compare those groups against the HCC Concept signatures in order to generate a score reflecting intersections between those groups. Specifically, for each HCC Concept signature, the list of ICD-10 and/or SNOMED parent concepts represented in the patient's problem list, as reflected in the problem list parent group, are compared against the ICD-10 and/or SNOMED parent concepts that make up that signature, and the system may identify the number and identity of the overlaps.

A comparison engine then may weigh the parent groups from the problem list against the HCC Concept signatures and create a prioritized list based upon goodness-of-fit scoring. Scoring takes into account how many parent groups define an HCC Concept, how many parent groups from the problem list match the HCC Concept, which HCC categories are not currently represented on the problem list, and what is a CMS-HCC normalization factor. Thus, e.g., a parent group that has 30 out of 30 concepts match may be considered a better match than a parent group that has 12 out of 12 concepts match. Conversely, a parent group that has 12 out of 12 concepts match with a first HCC Concept signature may be considered a better match than a second parent group that has 20 out of 40 concepts match with a second HCC Concept signature.

In particular, candidate HCC Concepts may presented in decreasing order of a goodness-to-fit score. In one instance, the system may apply a formula such as (a²+f)*h/b in order to generate the score, where:

a=number of problem list to HCC Concept parent matches;

b=number of HCC Concept parent groups identified;

f=a CMS-HCC diagnosis factor (“Community, FBDual, Disabled”); and

h=a binary option for excluding HCC Concepts already identified on the patient's problem list or HCC Concepts trumped by more significant HCC Concepts already identified on the patient's problem list, i.e., true=0, false=1.

As discussed above, the system also may exclude potential HCC Concepts that do not include a predetermined or user-defined minimum number of problem list matches, i.e., “a,” or parent group matches, i.e., “b.” For example, the system may exclude potential HCC Concepts for which a and/or b do not exceed 12 matches. In one instance, this may be accomplished by setting “h” to 0 if these minimum thresholds are not met.

Redundant HCC category diagnoses do not change a patient's Risk Adjustment Factor (RAF) score. For instance, a patient may have two diseases, e.g., “Type 1 diabetes mellitus with diabetic nephropathy” and “Type 1 diabetes mellitus with diabetic amyotrophy.” Those diseases may both be recognized as Type 1 diabetes mellitus with some complications of the same degree. As such, both may be found in the same HCC category, for instance HCC Category 18. In that instance, the system may not “credit” the user with both diseases with a change to the RAF score as compared to a similar patient with only one of the two diseases. Therefore, the display rule also may include a statement that if a particular HCC category appears on the problem list, all other diagnoses generated from the comparison engine with that same HCC category will be suppressed.

By using parent groups, the system is able to regulate the sensitivity and specificity of the suggestion results. In this regard, sensitivity may be the ability of a test to correctly identify those with a disease/problem, i.e., a true positive rate. In the equation above, sensitivity may be determined by the numerator that represents how many parent groups on a problem list match those belonging to an HCC Concept. Conversely, specificity measures a proportion of actual negatives that are correctly identified as such, e.g., a percentage of healthy people that are correctly identified as not having a disease/problem. In the equation above, specificity may be determined by the denominator that represents how many parent groups are used to define an HCC Concept. Additionally, prioritization for display takes into account the factor used to increase the reimbursement given to an organization for an HCC diagnosis.

Once the system identifies one or more HCC Concepts that are not already recognized in an electronic medical record or electronic health record or that are not considered less significant than HCC Concepts already recognized in that record, the system may generate a user interface with those HCC Concepts, e.g., ordered according to the heuristic discussed above or according to some other manner, and prompt a user to determine whether one or more of the HCC Concepts should be added to the record. If so, the system may accept the changes and revise the record to include the selected HCC Concept(s). In one instance, the user interface may categorize the list of possible HCC Concepts into one or more categories as part of the ordering heuristic, e.g., all diabetes-related HCC Concepts may be grouped together, all cardiovascular-related HCC Concepts may be grouped together, all neurological conditions may be grouped together, etc. Grouping may be accomplished, e.g., according to the same or a similar heuristic as that used to arrange the groupings of all HCCs. Selecting an HCC Concept from among those presented with respect to a certain group may cause the system to prevent a user from selecting another HCC Concept from that group. At the same time, however, such selection may not prevent the user from selecting an HCC Concept from a different group, e.g., a user selecting a diabetes-related HCC Concept may not be able to select a second diabetes-related HCC Concept but may be able to select a cardiovascular-related concept.

The system described herein may be used both to identify HCC Concepts in categories not previously identified in a patient record as well as to alter a current level of an HCC diagnosis. For example, in the former case, the analysis performed herein may identify to a user that the patient has sufficient problem list entries to warrant an identification of “Diabetes without Complication,” where no diabetes-related HCCs previously had been identified. Conversely, in the latter case, the patient's record prior to undertaking the optimization described herein may reflect an HCC of “Diabetes without Complication,” whereas after the analysis, it may reflect an HCC of “Diabetes with Chronic Complications.”

As set forth in greater detail herein, the present system and method are operable within a network of computer systems, with a plurality of computers each having a processor configured to operate EMR or EHR software accessible by one or more care providers to document patient encounters. In one aspect, each computer system operates the same EMR or EHR software. In another aspect, the computer systems may operate different EMR or EHR software packages that receive and/or store patient data in different ways. In this latter aspect, however, the various EMR or EHR software packages may interface with a common ontology such as an interface terminology in order to provide a common encoding mechanism for their respective sets of patient data.

As discussed above, the system may be used to evaluate individual patient records within an EMR or EHR. In another instance, the system may evaluate a plurality of patient records together in order to determine whether new HCCs may be relevant to one or more of the patients. In one aspect, this may be accomplished by performing the method described above once for each of the patients being considered. In another aspect, the system may generate one or more additional HCC signature reference tables using a patient's problem list and accompanying group of HCCs, where the patient's record reflects a particular HCC Concept and where the problem list may include one or more additional external ontology codes other than those used to encode the related HCC Concept parent signature.

While the first and second terminologies are referred to herein as “external,” it will be understood that this is to distinguish them from the interface terminology, in that they are separate terminologies and not, e.g., subsets of that terminology. Additionally, it will be appreciated that, while they are referred to as “external,” the interface terminology may not be “internal,” e.g., with regard to the EMR or EHR software. Instead, the interface terminology may be created and maintained by an entity separate from the EMR or EHR software provider. In this way, the same interface terminology may be used across multiple EMR or EHR software platforms, such that the system may be EMR or EHR-agnostic. Thus, rather than needing to develop a solution for each different EMR or EHR platform, the same system may be usable with each software package, thereby increasing the efficiency of developing and maintaining the system across multiple platforms.

While the foregoing written description of the invention enables one of ordinary skill to make and use what is considered presently to be the best mode thereof, those of ordinary skill will understand and appreciate the existence of variations, combinations, and equivalents of the specific exemplary embodiment and method herein. The invention should therefore not be limited by the above described embodiment and method, but by all embodiments and methods within the scope and spirit of the invention as claimed.

We claim:

1. A method, comprising:
translating a plurality of hierarchical condition category (HCC) codes into a plurality of HCC signatures, each HCC signature including one or more ICD-10 codes and/or one or more SNOMED codes;
extracting a plurality of ICD-10 codes and/or SNOMED codes out of an electronic medical record or electronic health record;
analyzing, using a comparison engine executed by a processor on a computer, the extracted codes against each of the HCC signatures;
identifying at least one HCC signature for which a minimum number of ICD-10 codes and/or SNOMED codes appear in the electronic medical record or electronic health record;
comparing the identified at least one HCC signature against HCC codes already recognized in the electronic medical record or electronic health record; and
if the electronic medical record or electronic health record does not include the identified at least one HCC signature, updating the electronic medical record or electronic health record to recognize an HCC code associated with at least one of the identified HCC signatures.

2. The method of claim 1, wherein the electronic medical record or electronic health record includes a problem list, wherein entries in the problem list are encoded with an interface terminology, and wherein the interface terminology includes a plurality of concepts mapped to a plurality of ICD-10 and/or SNOMED codes, the extracting step comprising:
identifying each of the ICD-10 codes and/or SNOMED codes mapped to concepts in the interface terminology encoding the entries in the problem list.

3. The method of claim 1, wherein the analyzing step comprises:
disregarding each extracted code that is not present in any of the plurality of HCC signatures.

4. The method of claim 1, wherein the updating step comprises:
generating a user interface including one or more HCC codes, each HCC code associated with a respective one of the identified at least one HCC signatures not included in the electronic medical record or electronic health record; and
receiving a user selection of one of the one or more HCC codes for updating the electronic medical record or electronic health record.

5. The method of claim 4, wherein the generating step comprises:
applying an weighting algorithm to each of the HCC codes to determine an order or ranking,
wherein the weighting algorithm ranks the HCC code higher if the HCC code has a greater number of extracted code to HCC signature matches.

6. The method of claim 1, wherein the extracting step comprises:
identifying one or more interface terminology concepts directly or indirectly mapped to elements of the electronic medical record or electronic health record; and
identifying each of the ICD-10 and/or SNOMED codes mapped to each of the one or more interface terminology concepts.

7. The method of claim 1, wherein the translating step comprises, for each HCC code:
identifying each ICD-10 and/or SNOMED code mapped to the HCC code;
identifying each ICD-10 and/or SNOMED code that is a hierarchical child of the identified ICD-10 and/or SNOMED code; and
including each identified hierarchical child in the HCC signature corresponding to the HCC code.

8. A method, comprising:
constructing a database comprising a plurality of HCC signatures, each HCC signature including one or more ICD-10 codes and/or one or more SNOMED codes;
deconstructing a patient problem list within an electronic medical record or electronic health record to generate an identification of one or more ICD-10 and/or SNOMED codes included within the patient problem list;
evaluating the identified one or more ICD-10 and/or SNOMED codes against each HCC signature to determine sufficient matches to one or more HCC signatures;
cross-checking HCC concepts related to the sufficiently-mapped one or more HCC signatures and eliminating HCC concepts already identified in the electronic medical record or electronic health record to generate a group of at least one non-eliminated, sufficiently-mapped HCC concepts; and
displaying the non-eliminated, sufficiently-mapped HCC concepts.

9. The method of claim 8, wherein the deconstructing step comprises:
identifying ICD-10 and/or SNOMED concepts mapping to one or more of the plurality of HCC signatures; and
ignoring ICD-10 and/or SNOMED concepts not mapping to one or more of the plurality of HCC signatures.

10. The method of claim 8, wherein the deconstructing step comprises:
identifying one or more interface terminology concepts directly or indirectly mapped to the patient problem list; and
determining the ICD-10 and/or SNOMED codes directly or indirectly mapped to the identified interface terminology concepts.

11. The method of claim 8, further comprising:
receiving a user selection of one of the displayed concepts; and
updating the electronic medical record or electronic health record to include the user-selected HCC concept.

12. The method of claim 11, wherein the updating step comprises:
adding a new HCC concept.

13. The method of claim 11, wherein the updating step comprises:
replacing an existing HCC concept with the user-selected HCC concept.

14. The method of claim 8, wherein sufficient matches is at least 12 matches.

15. The method of claim 8, wherein the displaying step comprises:
applying an weighting algorithm to each of the HCC codes to determine an order or ranking,
wherein the weighting algorithm ranks the HCC code higher if the HCC code has a greater number of identified code to HCC signature matches.

16. The method of claim 8, wherein the constructing step comprises, for each HCC code:
identifying each ICD-10 and/or SNOMED code mapped to the HCC code;
identifying each ICD-10 and/or SNOMED code that is a hierarchical child of the identified ICD-10 and/or SNOMED code; and
including each identified hierarchical child in the HCC signature corresponding to the HCC code.
